(19) United States
(12) Patent Application Publication
(10) Pub. No.: US 2023/0052196 A1
(43) Pub. Date: Feb. 16, 2023

(54) Head Stabilizer Device

(71) Applicant: Gary San Nicolas, Tacoma, WA (US)

(72) Inventor: Gary San Nicolas, Tacoma, WA (US)

(21) Appl. No.: 17/400,229

(22) Filed: Aug. 12, 2021

(51) Int. Cl. A61F 5/37; A61F 9/04

(52) U.S. Cl. CPC A61F 5/3707; A61F 9/04

(57) ABSTRACT

A head stabilizer device includes a hood having a front, a back, an interior surface, an exterior surface, and a perimeter edge extending between the interior surface and the exterior surface. The interior surface is concave. A tail is elongated having a first end coupled to the back of the hood wherein the tail extends downwardly from the back of the hood. The tail has a length for extending between a seat and buttocks of the person such that the tail is taut extending from the seat to the head such that an upper front section of the perimeter edge of the hood extends across the head of the person to inhibit the head from tilting forward.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to body part stabilizing devices and more particularly pertains to a new body part stabilizing device for holding a head in an upright position to allow a person to sleep comfortably while in a seated position.

### (2) Description of Related Art Including Information Disclosed Under 37 CFR 1.97 and 1.98

The prior art relates to body part stabilizing devices generally requiring a harness or straps which are secured to another body part or static structure.

### BRIEF SUMMARY OF THE INVENTION

An embodiment of the disclosure meets the needs presented above by generally comprising a hood having a front, a back, an interior surface, an exterior surface, and a perimeter edge extending between the interior surface and the exterior surface. The interior surface is concave. A tail is elongated having a first end coupled to the back of the hood wherein the tail extends downwardly from the back of the hood. The tail has a length for extending between a seat and buttocks of the person such that the tail is taut extending from the seat to the head such that an upper front section of the perimeter edge of the hood extends across the head of the person to inhibit the head from tilting forward.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a head stabilizer device according to an embodiment of the disclosure.

FIG. 2 is a right side view of an embodiment of the disclosure.

FIG. 3 is a rear view of an embodiment of the disclosure.

FIG. 4 is a top front side perspective view of an embodiment of the disclosure in use.

### DETAILED DESCRIPTION OF THE INVENTION

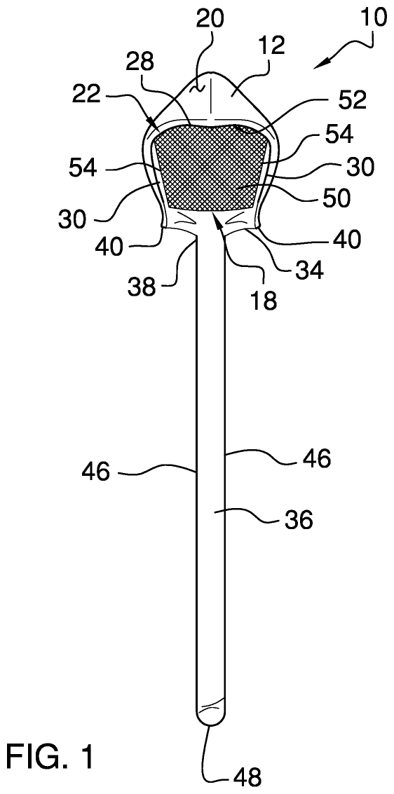

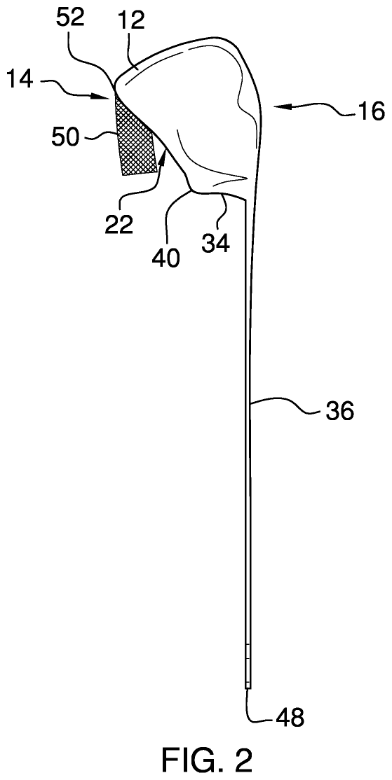

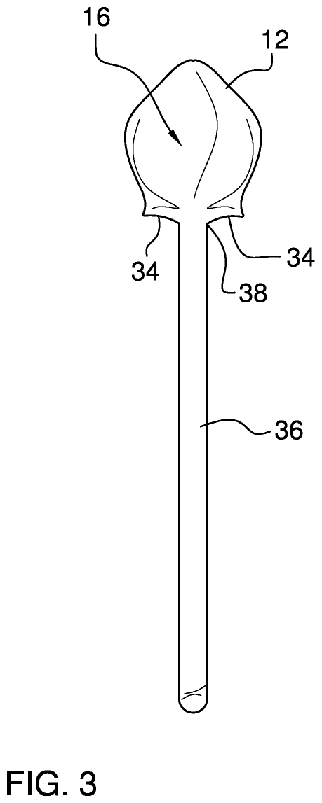

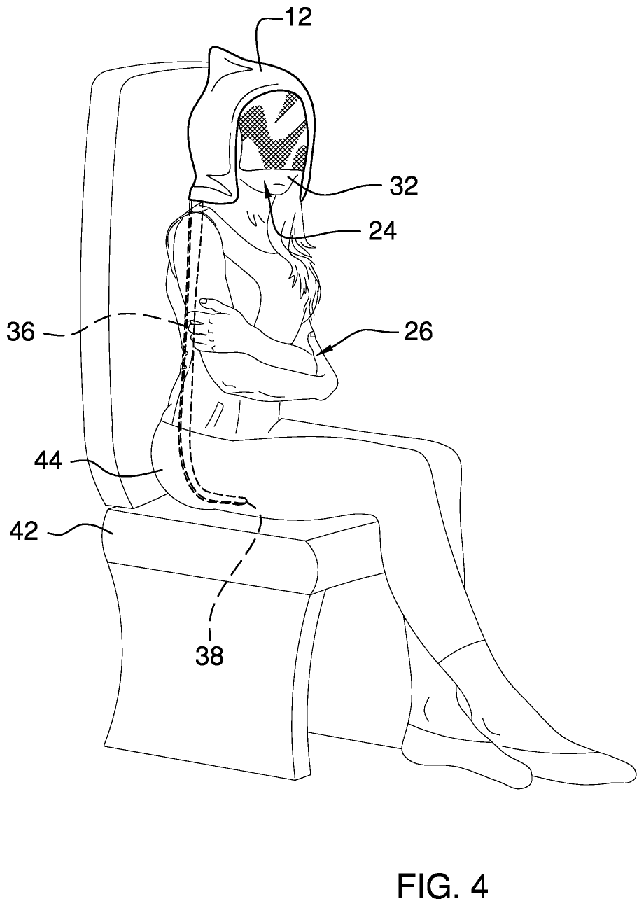

With reference now to the drawings, and in particular to FIGS. 1 through 4 thereof, a new body part stabilizing device embodying the principles and concepts of an embodiment of the disclosure and generally designated by the reference numeral 10 will be described.

As best illustrated in FIGS. 1 through 4, the head stabilizer device 10 generally comprises a hood 12 having a front 14, a back 16, an interior surface 18, an exterior surface 20, and a perimeter edge 22 extending between the interior surface 18 and the exterior surface 20. The interior surface 18 is concave wherein the hood 12 is configured for positioning on a head 24 of a person 26. The perimeter edge 22 has an upper front section 28 and a pair of front lateral sections 30. Each front lateral section 30 extends downwardly from a respective side of the upper front section 28. Thus, the upper front section 28 and the pair of front lateral sections 30 are configured to extend around a face 32 of the person 26. The perimeter edge 22 of the hood 12 further has a back bottom section 34. The back bottom section 34 is curved extending downwardly and inwardly towards a center of the back bottom section 34.

A tail 36 is elongated and has a first end 38 coupled to the back 16 of the hood 12 wherein the tail 36 extends downwardly from the back 16 of the hood 12. More specifically, the tail 36 extends from the back bottom section 34 of the perimeter edge 22 of the hood 12. The tail 36 is centered between outer ends 40 of the back bottom section 34 of the perimeter edge 22 of the hood 12. The tail 36 has a length wherein the tail 36 is configured for extending to be positioned between a seat 42 and buttocks 44 of the person 26 such that the tail 36 is taut extending from the seat 42 to the head 24 of the person 26. Thus, the upper front section 28 of the perimeter edge 22 of the hood 12 extends across the head 24 of the person 26 to inhibit the head 24 from tilting forward. The tail 36 has a pair of lateral edges 46. The lateral edges 46 of the tail 36 are parallel to each other. The lateral edges 46 of the tail 36 are spaced between 2.0 and 4.0 inches apart to provide sufficient width to lay comfortably along a spine of the person 26 while not being bulky for storage of the device 10 when not in use. The spacing between the lateral edges 46 may be 3.0 inches apart. A distal edge 48 of the tail 36 relative to the hood 12 is arcuate extending between the pair of lateral edges 46 of the tail 36. The length of the tail 36 may correspond to a length of an average sized human from approximately the base of the neck to 2.0-5.0 inches under the buttocks. The device 10 may also be provided with the length of the tail 36 being provided corresponding to general sizes of persons based on torso length such as short, medium, long, extra long, small, large, or the like.

A mask 50 is coupled to the hood 12. The mask 50 is positionable between the front lateral sections 30 of the perimeter edge 22 whereby the mask 50 is configured for covering the face 32 of the person 26 when the hood 12 is positioned on the head 24 of the person 26. The mask 50 is coupled to the hood 12 along a top edge 52 of the mask 50. The mask 50 has lateral edges 54. The lateral edges 54 of the mask 50 are free whereby the mask 50 hangs freely down from the upper edge 52 of the mask 50. The mask may be partially or fully opaque to provide privacy and cover eyes of the person in the manner of a sleep mask but without straps or tight fitting over the face 24 of the person 26 to enhance comfort.

In use, the hood 12 is positioned on the head 24 of the person 26. The tail 36 is extended along the back of the person 26 and under the buttocks 44 such that the tail 36 is pressed onto the seat 42 by the weight of the person 26. The tail 36 may be adjusted such that the hood 12 exerts a comfortable amount of pressure along the upper front section 28 to the head 24 of the person 26 to inhibit the head 24 from tilting further forward. The mask 50 is positionable to extend over the face 24 of the person 26 allowing the person 26 to sleep with the head 24 being supported in an upright position by the device 10.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A head stabilizer device comprising:
a hood having a front, a back, an interior surface, an exterior surface, and a perimeter edge extending between the interior surface and the exterior surface, the interior surface being concave wherein said hood is configured for positioning on a head of a person; and
a tail, said tail being elongated, said tail having a first end coupled to said back of said hood wherein said tail extends downwardly from said back of said hood, said tail having a length wherein said tail is configured for extending between a seat and buttocks of the person such that said tail is taut extending from the seat to the head of the person whereby an upper front section of said perimeter edge of said hood extends across the head of the person to inhibit said the head from tilting forward.

2. The head stabilizer device of claim 1, wherein said perimeter edge has a pair of front lateral sections, each front lateral section extending downwardly from a respective side of said upper front section wherein upper front section and said pair of front lateral sections are configured to extend around a face of the person.

3. The head stabilizer device of claim 2, further comprising a mask coupled to said hood, said mask being positionable between said front lateral sections of said perimeter edge whereby said mask is configured for covering the face of the person when the hood is positioned on the head of the person.

4. The head stabilizer device of claim 1, further comprising said tail having a pair of lateral edges, said lateral edges of said tail being parallel to each other.

5. The head stabilizer device of claim 4, further comprising a distal edge of said tail relative to said hood being arcuate extending between said pair of lateral edges.

6. The head stabilizer device of claim 1, further comprising said perimeter edge of said hood having a back bottom section, said back bottom section being curved extending downwardly and inwardly towards a center of said back bottom section.

7. The head stabilizer device of claim 6, further comprising said tail extending from said back bottom section of said perimeter edge of said hood.

8. The head stabilizer device of claim 7, further comprising said tail being centered between outer ends of said back bottom section of said perimeter edge of said hood.

9. The head stabilizer device of claim 3, further comprising said mask being coupled to said hood along a top edge of said mask.

10. The head stabilizer device of claim 9, further comprising lateral edges of said mask being free whereby said mask hangs freely down from said upper edge of said mask.

11. The head stabilizer device of claim 1, wherein said tail has a minimum width greater than 2.0 inches and a maximum width less than 4.0 inches.

12. The head stabilizer device of claim 4, wherein said lateral edges of said tail are spaced between 2.0 inches and 4.0 inches apart.

13. The head stabilizer device of claim 4, wherein said lateral edges of said tail are spaced 3.0 inches apart.

14. A head stabilizer device comprising:
a hood having a front, a back, an interior surface, an exterior surface, and a perimeter edge extending between the interior surface and the exterior surface, the interior surface being concave wherein said hood is configured for positioning on a head of a person, said perimeter edge having an upper front section and a pair of front lateral sections, each front lateral section extending downwardly from a respective side of said upper front section wherein upper front section and said pair of front lateral sections are configured to extend around a face of the person, said perimeter edge of said hood having a back bottom section, said back bottom section being curved extending downwardly and inwardly towards a center of said back bottom section;
a tail, said tail being elongated, said tail having a first end coupled to said back of said hood wherein said tail extends downwardly from said back of said hood, said tail extending from said back bottom section of said perimeter edge of said hood, said tail being centered between outer ends of said back bottom section of said perimeter edge of said hood, said tail having a length wherein said tail is configured for extending between a seat and buttocks of the person such that said tail is taut extending from the seat to the head of the person whereby said upper front section of said perimeter edge of said hood extends across the head of the person to inhibit said the head from tilting forward, said tail having a pair of lateral edges, said lateral edges of said tail being parallel to each other, wherein said lateral edges of said tail are spaced 3.0 inches apart, a distal edge of said tail relative to said hood being arcuate extending between said pair of lateral edges of said tail; and
a mask coupled to said hood, said mask being positionable between said front lateral sections of said perimeter edge whereby said mask is configured for covering the face of the person when the hood is positioned on the head of the person, said mask being coupled to said hood along a top edge of said mask, said mask having lateral edges, said lateral edges of said mask being free whereby said mask hangs freely down from said upper edge of said mask.
